(19) United States
(12) Patent Application Publication
(10) Pub. No.: US 2006/0159693 A1
(43) Pub. Date: Jul. 20, 2006

(54) Idiotypic vaccine

(76) Inventor: Robyn Lynne Ward, New South Wales (AU)

Correspondence Address: KING & SPALDING LLP, 1180 PEACHTREE STREET, ATLANTA, GA, 30309, US

(21) Appl. No.: 10/546,901

(22) PCT Filed: Feb. 23, 2004
(86) PCT No.: PCT/AU04/00225

§ 371 Date: Aug. 19, 2005

(30) Foreign Application Priority Data
Feb. 21, 2003 (AU) 2003900767

(51) Int. Cl. A61K 39/00

(52) U.S. Cl. 424/185.1

(57) ABSTRACT

The present invention relates to idiotypic vaccine compositions for use in inducing immunity to p53. The invention preferably relates to a vaccine composition comprising a pharmaceutically acceptable carrier and at least one peptide, wherein the at least one peptide is selected from the group consisting of X₁-LLQALKH-Y₁, X₂-FIRKAYGAATAYAASKKG-Y₂ and X₃-MQGLQTPYT-Y₃ in which X₁, X₂, X₃, Y₁, Y₂ and Y₃ are independently either absent or an amino acid sequence of preferably less than 10 amino acids which provides a framework for the specified peptide.

### FIELD OF THE INVENTION

The present invention relates to idiotypic vaccine compositions for use in inducing immunity to p53.

### BACKGROUND OF THE INVENTION

Mutation of the p53 tumor-suppressor gene occurs in almost 50% of human cancers, including colon (65%), lung (70%), stomach (45%), breast (30%) and head and neck (60%). p53 has three functional domains: an acidic N-terrninal domain characteristic of transcription factors, a regulatory C-terminal domain and a central DNA-binding domain. Mutated p53 protein loses its normal function in regulating the cell cycle and apoptosis. Mutated p53 is not appropriately degraded and therefore accumulates in the cell cytoplasm and nucleus.

p53 is considered to be an excellent tumor associated antigen for active immunotherapy because: (1) Overexpression of p53 results in the high level display of p53 peptides on the surface of tumor cells in association with human Class I HLA molecules; (2) p53 is one of the most well-characterised antigens, its gene sequence is known, and the three-dimensional structure of a number of key regions of the protein have been determined allowing structural definition and mapping of the relevant unique antigenic sites on p53 that offer useful targets for humoral and cellular immunity. Another good reason to target the molecule is that 1) p53 peptides are not displayed on normal cells; 2) T cells specific for p53 exist in humans. Unfortunately a number of studies have demonstrated that p53 is not a very potent immunogen probably because the molecule is a self-protein. If tolerance to p53 can be broken there is a theoretical possibility that vaccination will trigger a strong autoimmune reaction useful in retarding tumour progression. An alternative to the use of p53 fragments, is to use antigenic mimics which are devoid of any undesired properties that cause immunological tolerance and yet retain the ability to induce p53 specific immune responses.

In this invention, we provide an idiotypic vaccine comprising CDRs of human antibodies directed against p53.

The idiotypic network hypothesis first proposed by Lindemann in 1973 and Jerne in 1974 described the immune system as a network of interacting antibodies and lymphocytes. In this hypothesis, an antibody Ab1 can be used to generate a series of anti-idiotype antibodies against Ab1, termed Ab2. Some of these Ab2 molecules, termed Ab2β, can fit into the paratopes of Ab1 and act as a functional mimic of the three-dimensional structure of the tumor-associated antigen (TAA) identified by Ab1. The Ab2β can in turn induce specific anti-anti-idiotype antibodies (Ab3) and T cells (T3) that recognise the original tumor-associated antigen identified by Ab1.

The use of anti-idiotype antibody (Ab2α) as a vaccination strategy has several major advantages: (1) It is safer to use as it does not involve the use of tumor-derived material to induce anti-tumor immunity; (2) it could represent an effective method of breaking tolerance to self tumor antigens, as the epitope structure is now transformed into an idiotype determinant and is expressed in a different molecular environment; (3) anti-idiotype antibody may have a higher affinity than the original antigen to bind to class I HLA molecules. This will favour the expression on antigen-presenting cells (APC) that process the anti-idiotype and favours T-cell activation; (5) when human anti-idiotypic antibodies are used, they may have the advantage of stimulating more efficiently human immune effector cells and in particular T cells. Recently, several clinical trials have showed that anti-idiotype antibody is very well tolerated.

In International Patent Application No. WO 00/56770 the present inventors disclose a number of human antibodies directed against p53. In addition the amino acid sequence of these antibodies together with the DNA sequences encoding these antibodies is provided. The disclosure of this application is included herein by reference.

### SUMMARY OF THE INVENTION

In a first aspect the present invention provides an idiotypic vaccine composition, the vaccine composition comprising a pharmaceutically acceptable carrier and at least one peptide, wherein the at least one peptide is selected from the group consisting of X₁-LLQALKH-Y₁, X₂-FIRSKAYGAATAYAASMKG-Y₂ and X₃-MQGLQTPYT-Y₃, in which X1, X2, X3, Y1, Y2 and Y3 are independently either absent or an amino acid sequence of preferably less than 10 amino acids which provides a framework for the specified peptide.

In a second aspect the present invention provides an idiotypic vaccine composition, the composition comprising a pharmaceutically acceptable carrier and at least one peptide, the at least one peptide being characterised in that it competes with a peptide selected from the group consisting of AVYYCLLQALKHWGQGT, LEWVGFIRSKAYGAATAYAASMKGRVTI, and GVYYCMQGLQTPYTFGEGT for binding to p53.

In a third aspect the present invention provides an idiotypic vaccine composition, the composition comprising a pharmaceutically acceptable carrier and at least one peptide, the at least one peptide being characterised in that an antibody raised against the peptide reacts with at least one peptide selected from the group consisting of AVYYCLLQALKHWGQGT, LEWVGFIRSKAYGAATAYAASMKGRVTI, and GVYYCMQGLQTPYTFGEGT.

In a fourth aspect, the present invention provides an idiotypic vaccine composition, the vaccine comprising a pharmaceutically acceptable carrier and at least one DNA molecule, the DNA molecule comprising a sequence encoding at least one peptide, wherein the at least one peptide is selected from the group consisting of X₁-LLQALKH-Y₁, X₂-FIRSKAYGAATAYAASMKG-Y₂ and X₃-MQGLQTPYT-Y₃, in which X1, X2, X3, Y1, Y2 and Y3 are independently either absent or an amino acid sequence of preferably less than 10 amino acids which provides a framework for the specified peptide.

In a fifth aspect, the present invention provides an idiotypic vaccine composition, the composition comprising a pharmaceutically acceptable carrier and at least one DNA molecule, the DNA molecule comprising a sequence encoding at least one peptide, the at least one peptide being characterised in that it competes with a peptide selected from the group consisting of AVYYCLLQALKHWGQGT, LEWVGFIRSKAYGAATAYAASMKGRVTI, and GVYYCMQGLQTPYTFGEGT for binding to p53.

In a sixth aspect the present invention provides an idiotypic vaccine composition, the composition comprising a pharmaceutically acceptable carrier and at least one DNA molecule, the DNA molecule comprising a sequence encoding at least one peptide, the at least one peptide being characterised in that an antibody raised against the peptide reacts with at least one peptide selected from the group consisting of AVYYCLLQALKHWGQGT, LEWVGFIRSKAYGAATAYAASMKGRVTI, and GVYYCMQGLQTPYTFGEGT.

In a seventh aspect the present invention consists in a method of inducing an anti-p53 idiotypic response in a subject, the method comprising administering to the subject the composition according to any one of the first, second, third, fourth, fifth or sixth aspects of the invention.

In an eight aspect the present invention provides a method of inducing immunity against a disease caused by expression of mutant p53, the method comprising administering to the subject the composition according to any one of the first, second, third, fourth, fifth or sixth aspects of the invention.

### BRIEF DESCRIPTION OF THE FIGURES

FIG. 1. Humoral responses to individual vaccine peptides as measured by ELISA over the course of the vaccination schedule. Individual graphs show the results for one trial subject (subject 012—A, G and I; subject 014—B; subject 015—C, E and H and subject 016—D, F and J). The serum reactivity with peptide 2 is shown in figures A-D and I peptide 5 in figures E, F and J and peptide 8 is shown in G and H. Individual biotinylated vaccine peptides (5 μg/ml) were bound to streptavidin-coated plates (5 μg/ml) and serum binding antibodies were detected using either a goat anti-human IgG, IgA and IgM-specific secondary alkaline-phosphatase conjugate (0.12 μg/ml) (A-H) or a panel of murine anti-human IgA-, IgG1-, IgG2-, IgG3- and IgG4-, IgM specific antibodies (0.25 μg/ml) followed by a goat anti-mouse IgG alkaline phosphatase conjugate (0.12 μg/ml) (I and J). Serum dilutions were 1:50, except A, 1:100; G, 1:25; H, 1:25. Visit 1—baseline; Visit 2—1 month after vaccination 1; Visit 3—1 month after vaccination 2; Visit 4—1 month after vaccination 3; Visit 5—1 month after vaccination 4; Visit 6—2 months after vaccination 4. Mean of triplicates±one standard deviation is shown.

FIG. 2. Serum antibody responses to the individual vaccine peptides (A) and to p53 (B) in one of two sheep immunized with the pooled vaccine as measured by ELISA. A. Individual biotinylated vaccine peptides (5 μg/ml) were bound to streptavidin-coated plates (5 μg/ml) and serum binding antibodies present 10 days after the boost were detected with an alkaline phosphatase conjugated donkey anti-sheep IgG-specific secondary antibody (0.12 μg/ml). B. Purified recombinant p53 (10 μg/ml) was bound to plates and serum binding antibodies present before (pre-immune serum) and after (post-immune serum) immunization, were detected as above. Mean of triplicates±one standard deviation is shown.

FIG. 3. Humoral response to p53 in trial subject 012 measured over the course of the vaccination schedule by ELISA. Purified recombinant p53 (10 μg/ml) was bound to plates and serum binding antibodies were detected with either a goat anti-human IgG, IgA and IgM-specific secondary alkane-phosphatase conjugate (0.12 μg/ml) (A) or a panel of murine anti-human isotype specific antibodies (0.25 μg/ml) followed by a goat anti-mouse IgG alkaline phosphatase conjugate (0.12 μg/ml) (B). Data shown uses serum diluted 1:100, while reactivity was still detected at dilutions of 1:400. The relative serum concentration was calculated by dividing the concentration of a pool of two positive control sera giving the equivalent absorbance at 410 nm to the test sample, by the concentration of the test sample. Visit 1—baseline; Visit 2—1 month after vaccination 1; Visit 3—1 month after vaccination 2; Visit 4—1 month after vaccination 3; Visit 5—1 month after vaccination 4; Visit 6—2 months after vaccination 4. Mean of triplicates±one standard deviation is shown.

FIG. 4. Cell-mediated response to peptide 5 in trial subject 016, measured over the course of the vaccination schedule. Patient PBMCs were stimulated with 10 μg/ml peptide 5, incubated in vitro for 6 days, pulsed with ³H-thymidine for 18 hours and then proliferation was assessed by ³H-thymidine incorporation. Visit 1—baseline; Visit 2—1 month after vaccination 1; Visit 3—1 month after vaccination 2; Visit 4—1 month after vaccination 3; Visit 5—1 month after vaccination 4; Visit 6—2 months after vaccination 4.

FIG. 5. Cell-mediated response to the vaccine in two subjects, 016 (A and B) and 017 (C and D) measured at baseline time points (A and C) and 1 month after vaccination 3 (B) or 4 (D). CFSE-FITC stained subject PBMCs were stimulated with 50 μg/ml of pooled vaccine and incubated in vitro for 6 days. CD8, CD71 and CD3 markers were detected using fluorescein-conjugated antibodies. Percentages of CD3- , CD4- and CD71-positive cells that have specifically proliferated in response to in vitro stimulation with the vaccine are shown.

### DETAILED DESCRIPTION

The present inventors have demonstrated that immunisation with particular peptides derived from the CDRs of human anti-p53 antibodies results in the generation of Ab2. As part of the idiotype cascade Ab2 will lead to the generation of Ab3, i.e. antibodies directed against p53. The CDRs identified and selected are unique in that they result in the generation of Ab2 (antibody that represents a p53 structural mimic) that in turn results in immune responses (humoral and cell mediated) to regions on the three dimensional structure of p53 valuable in practicing tumour cell killing and the invention. There will also be regions on p53, defined by recognition by other CDRs from human monoclonal antibodies (Ab1s) which although immunogenic, may produce immune responses (Ab2s) but would fail to achieve tumoricidal activity. Human monoclonal antibodies (Ab1s) can be created to almost any region of p53, as processing of the molecule during its clearance in the body exposes many epitopes that may not be immunologically relevant for targeting immunity in the form of a vaccine. A key to the invention is the identification of p53 sequence regions with Ab1s (the human monoclonal antibodies) that provide a site to which a successful anti-idiotypic cascade can be targeted. Thus an important aspect of the invention is the selection of CDRs on Ab1s (as defined by particular p53 sequences to which they bind) that in turn elicit immunity, via the anti-idiotypic cascade, to regions of the mutant p53 molecule that are exposed for recognition by CTLs.

As will be readily appreciated, while the results described herein have been achieved using peptides of particular sequence, similar results may be achieved using peptides of differing sequence. The present invention also extends to analogues of the peptides of the present invention. The critical factor is that the peptide antigen elicits substantially the same Ab2 response as at least one of the specified peptides. Accordingly, the essential characteristic of the peptide antigen is that it elicits an antibody that reacts with at least one of the specified peptide antigens.

Accordingly, in a first aspect the present invention provides an idiotypic vaccine composition the vaccine composition comprising a pharmaceutically acceptable carrier and at least one peptide, wherein the at least one peptide is selected from the group consisting of X₁-LLQALKH-Y₁, X₂-FIRSKAYGAATAYAASMKG-Y₂ and X₃-MQGLQTPYT-Y₃, in which X1, X2, X3, Y1, Y2 and Y3 are independently either absent or an amino acid sequence of preferably less than 10 amino acids which provides a framework for the specified peptide.

In a preferred embodiment of the present invention
X₁ is absent or is AVYYC
X₂ is absent or is LEWVG
X₃ is absent or is GVYYC
Y₁ is absent or is WGQGT
Y₂ is absent or is RVTI
Y₃ is absent or is FGEGT.

In a further preferred embodiment the composition comprises at least one peptide selected from the group consisting of AVYYCLLQALKHWGQGT, LEWVGFIRSKAYGAATAYAASMKGRVTI, and GVYYCMQGLQTPYTFGEGT.

In a further preferred embodiment the composition comprises at least 2 of, and more preferably, all 3 of the peptides AVYYCLLQALKHWGQGT, LEWVGFIRSKAYGAATAYAASMKGRVTI, and GVYYCMQGLQTPYTFGEGT.

In another preferred embodiment the composition further comprises at least one peptide selected from the group consisting of LEWMGIINPSGGSANYAPKFKGRLTMS, KLLIHWASTRESGVPDR, AGLFCQQYYTTPLTFGGGT, YFCSRVKAGGPDYWGQGT and LLIYLGSTRASGVPDR.

In a second aspect the present invention provides an idiotypic vaccine composition, the composition comprising a pharmaceutically acceptable carrier and at least one peptide, the at least one peptide being characterised in that it competes with a peptide selected from the group consisting of AVYYCLLQALKHWGQGT, LEWVGFIRSKAYGAATAYAASMKGRVTI, and GVYYCMQGLQTPYTFGEGT for binding to p53.

By “compete” it is meant that in the presence of the peptide at the same concentration, the binding of AVYYCLLQALKHWGQGT, LEWVGFIRSKAYGAATAYAASMKGRVTI, or GVYYCMQGLQTPYTFGEGT to p53 is reduced by at least 50%. The peptide will bind to p53 with equivalent, better or up to two orders of magnitude weaker affinity.

Competition may be measured in any of a number of ways well known to persons skilled in the art. Preferably the level of competition is measured by labelling AVYYCLLQALKHWGQGT, LEWVGFIRSKAYGAATAYAASMKGRVTI, or GVYYCMQGLQTPYTFGEGT and measuring the level of binding to p53 in the presence or absence of the test peptide.

In a third aspect the present invention provides an idiotypic vaccine composition, the composition comprising a pharmaceutically acceptable carrier and at least one peptide, the at least one peptide being characterised in that an antibody raised against the peptide reacts with at least one peptide selected from the group consisting of AVYYCLLQALKHWGQGT, LEWVGFIRSKAYGAATAYAASMKGRVTI, and GVYYCMQGLQTPYTFGEGT.

By “reacts” it is meant that the antibody raised against the peptide binds to at least one peptide selected from AVYYCLLQALKHWGQGT, LEWVGFIRSKAYGAATAYAASMKGRVTI, and GVYYCMQGLQTPYTFGEGT.

In a preferred embodiment of the second and third aspects of the present invention, the peptide is selected from the group consisting of X₁-LLQALKH-Y₁, X₂-FIRSKAYGAATAYAASMKG-Y₂ and X₃-MQGLQTPYT-Y_{3,} in which X1, X2, X3, Y1, Y2 and Y3 are independently either absent or an amino acid sequence of preferably less than 10 amino acids which provides a framework for the specified peptide.

In a further preferred embodiment of the second and third aspects of the present invention
X₁ is absent or is AVYYC
X₂ is absent or is LEWVG
X₃ is absent or is GVYYC
Y₁ is absent or is WGQGT
Y₂ is absent or is RVTI
Y₃ is absent or is FGEGT.

In a further preferred embodiment of the second and third aspects of the present invention the composition comprises at least one peptide selected from the group consisting of AVYYCLLQALKHWGQGT, LEWVGFIRSKAYGAATAYAASMKGRVTI, and GVYYCMQGLQTPYTFGEGT.

In a further preferred embodiment of the second and third aspects of the present invention the composition comprises at least 2 of, and more preferably, all 3 of the peptides AVYYCLLQALKHWGQGT, LEWVGFIRSKAYGAATAYAASMKGRVTI, and GVYYCMQGLQTPYTFGEGT.

In another preferred embodiment of the second and third aspects of the present invention the composition further comprises at least one peptide selected from the group consisting of LEWMGIINPSGGSANYAPKFKGRLTMS, KLLIHWASTRESGVPDR, AGLFCQQYYTTPLTFGGGT, YFCSRVKAGGPDYWGQGT and LLIYLGSTRASGVPDR.

In a further preferred embodiment of the first, second and third aspects of the present invention the composition further comprises an adjuvant

The ideal immune response against cancer is generally considered to involve the induction of cytotoxic T cells and a Th1 helper response. Despite the availability of agents that facilitate the skewing of the immune response towards this outcome the selection of adjuvant for cancer vaccines involves a level of routine experimentation in selection of the appropriate adjuvant.

In addition to the adjuvant it is often the case that a small peptide (generally less than 30 amino acids) is linked to a larger molecule (the carrier) to improve its immunogenicity. The selection of suitable carrier and adjuvant is key to the preparation of a safe and acceptable vaccine for human use.

In the trial set out below granulocyte-macrophage colony stimulating factor (GM-CSF) was used as an adjuvant. This cytokine is thought to be useful by virtue of its ability to mobilize antigen presenting cells and up-regulation of MHC class I and II molecules. In the context of peptide vaccines GM-CSF tends to induce a mixed Th1/Th2 response. Other cytokine adjuvants such as IL-2 and IL-12 may also be used, however, these have also been associated with excessive adverse effects and may not be suitable for a vaccine that reaches routine clinical use.

Other commonly used adjuvants have a largely undefined mechanism of action and may exert their effects purely by physical means such as prolonging antigen presentation and enhancing antigen localisation. These include miscellaneous oil-emulsion technologies, ISCOMS, alum (aluminium hydroxide and aluminium phosphate), and liposomal delivery systems. Some adjuvants appear to act via pathogen-recognition receptors (PRRs) to induce various co-stimulatory events necessary for the immune response. These include CpG oligonucleotides, lipopolysaccharide (LPS), the muramyl dipeptides (such as N-acetyl-muramyl-L-threonyl-D-isoglutamine (thr-MDP), N-acetyl-nor-muramyl-L-alanyl-D-isoglutamine (CGP 11637, referred to as nor-MDP) and bacterial toxins such as cholera toxin. In the latter case the peptide could be conjugated to a bacterial toxin, a technique that is being trialed in human gastrointestinal malignancy with G17-DT, a conjugate of gastrin with diphtheria-toxin. All of these agents, to some degree influence polarisation of the immune response.

As with diphtheria toxin and cholera toxin the peptides may be conjugated with other bacterial toxins such as tetanus toxoid or proteins such as KLH (keyhole limpet haemocyanin).

Additional examples of adjuvants which may be effective include but are not limited to N-acetylmuramyl-L-alanyl-D-isoglutaminyl-L-alanine-2-(1′-2′-dipalmit-oyl-sn-glycero-3hydroxyphosphoryloxy)ethylamine (CGP 19835A, referred to as MTP-PE), and RIBI, which contain three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminium potassium sulfate (alum), beryllium sulfate, silica, kaolin, carbon, water-in-oil emulsions, oil-in-water emulsions, Coxynebadenum prarum (Propionobacteiun acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). It is particularly preferred to include adjuvants which promote helper T cell responses, such as diphtheria, pertussis and tetanus toxins and ovalbumin. Other preferred adjuvants include immune stimulatory complexes (ISCOMs) which are small micelles of detergent such as Quil A. The immunogens of the invention may be present within the micelles which can fuse with antigen-presenting cells, allowing the immunogen to enter the cytosol.

Polypeptides of the present invention, especially peptides, may also be prepared as self-adjuvanting peptides by conjugation to fatty acids, for example as described in WO93/02706.

It is presently preferred, however, that the adjuvant is GM-CSF.

In a fourth aspect, the present invention provides an idiotypic vaccine composition, the vaccine comprising a pharmaceutically acceptable carrier and at least one DNA molecule, the DNA molecule comprising a sequence encoding at least one peptide, wherein the at least one peptide is selected from the group consisting of X₁-LLQALKH-Y₁, X₂-FIRSKAYGAATAYAASMKG-Y₂ and X₃-MQGLQTPYT-Y3, in which X1, X2, X3, Y1, Y2 and Y3 are independently either absent or an amino acid sequence of preferably less than 10 amino acids which provides a framework for the specified peptide.

In a preferred embodiment of the fourth aspect of the present invention
X₁ is absent or is AVYYC
X₂ is absent or is LEWVG
X₃ is absent or is GVYYC
Y₁ is absent or is WGQGT
Y₂ is absent or is RVTI
Y₃ is absent or is FGEGT.

In a further preferred embodiment of the fourth aspect of the present invention the composition comprises a DNA molecule encoding at least one peptide selected from the group consisting of AVYYCLLQALKHWGQGT, LEWVGFIRSKAYGAATAYAASMKGRVTI, and GVYYCMQGLQTPYTFGEGT.

In a further preferred embodiment of the fourth aspect of the present invention the DNA molecule encodes at least 2 and more preferably all 3 of the peptides AVYYCLLQALKHWGQGT, LEWVGFIRSKAYGAATAYAASMKGRVTI, and GVYYCMQGLQTPYTFGEGT.

In a further preferred embodiment of the fourth aspect of the present invention the DNA molecule comprises a further sequence encoding GM-CSF.

In another preferred embodiment of the fourth aspect of the present invention the DNA molecule comprises a further sequence encoding at least one peptide selected from the group consisting of LEWMGIINPSGGSANYAPKFKGRLTMS, KLLIHWASTRESGVPDR, AGLFCQQYYTTPLTFGGGT, YFCSRVKAGGPDYWGQGT and LLIYLGSTRASGVPDR.

In a fifth aspect the present invention provides an idiotypic vaccine composition, the composition comprising a pharmaceutically acceptable carrier and at least one DNA molecule, the DNA molecule comprising a sequence encoding at least one peptide, the at least one peptide being characterised in that it competes with a peptide selected from the group consisting of AVYYCLLQALKHWGQGT, LEWVGFIRSKAYGAATAYAASMKGRVTI, and GVYYCMQGLQTPYTFGEGT for binding to p53.

In a sixth aspect the present invention provides an idiotypic vaccine composition, the composition comprising a pharmaceutically acceptable carrier and at least one DNA molecule, the DNA molecule comprising a sequence encoding at least one peptide, the at least one peptide being characterised in that an antibody raised against the peptide reacts with at least one peptide selected from the group consisting of AVYYCLLQALKHWGQGT, LEWVGFIRSKAYGAATAYAASMKGRVTI, and GVYYCMQGLQTPYTFGEGT.

In a preferred embodiment of the fifth and sixth aspects of the invention, the peptide encoded by the DNA molecule is selected from the group consisting of X₁-LLQALKH-Y₁, X₂-FIRSKAYGAATAYAASMKG-Y₂ and X₃-MQGLQTPYT-Y_{3,} in which X1, X2, X3, Y1, Y2 and Y3 are independently either absent or an amino acid sequence of preferably less than 10 amino acids which provides a framework for the specified peptide.

In a preferred embodiment of the fifth and sixth aspects of the present invention
X₁ is absent or is AVYYC
X₂ is absent or is LEWVG
X₃ is absent or is GVYYC
Y₁ is absent or is WGQGT
Y₂ is absent or is RVTI
Y₃ is absent or is FGEGT.

In a further preferred embodiment of the fifth and sixth aspects of the present invention the composition comprises a DNA molecule encoding at least one peptide selected from the group consisting of AVYYCLLQALKHWGQGT, LEWVGFIRSKAYGAATAYAASMKGRVTI, and GVYYCMQGLQTPYTFGEGT.

In a further preferred embodiment of the fifth and sixth aspects of the present invention the DNA molecule encodes at least 2 and more preferably all 3 of the peptides AVYYCLLQALKHWGQGT, LEWVGFIRSKAYGAATAYAASMKGRVTI, and GVYYCMQGLQTPYTFGEGT.

In a further preferred embodiment of the fifth and sixth aspects of the present invention the DNA molecule comprises a further sequence encoding GM-CSF.

In another preferred embodiment of the fifth and sixth aspects of the present invention the DNA molecule comprises a further sequence encoding at least one peptide selected from the group consisting of LEWMGIINPSGGSANYAPKFKGRLTMS, KLLIHWASTRESGVPDR, AGLFCQQYYTTPLTFGGGT, YFCSRVKAGGPDYWGQGT and LLIYLGSTRASGVPDR.

As will be recognised the composition of the fourth, fifth and sixth aspects of the present invention is for use in DNA vaccination.

The ability of direct injection of non-replicating plasmid DNA coding for viral proteins to elicit protective immune responses in laboratory and preclinical models has created increasing interest in DNA immunisation. A useful review of DNA vaccination is provided in Donnelly et al¹, the disclosure of which is incorporated herein by reference.

DNA vaccination involves the direct in vivo introduction of DNA encoding an antigen into tissues of a subject for expression of the antigen by the cells of the subject's tissue. DNA vaccines are described in U.S. Pat. No. 5,939,400, U.S. Pat. No. 6,110,898, WO 95/20660 and WO 93/19183, the disclosures of which are hereby incorporated by reference in their entireties. The ability of directly injected DNA that encodes an antigen to elicit a protective immune response has been demonstrated in numerous experimental systems (see, for example, Conry et al², Cardoso et al³, Cox et al⁴, Davis et al⁵, Sedegah et al⁶, Montgomery et al⁷, Ulmer et al⁸, Wang et al⁹, Xiang et al¹⁰, Yang et al¹¹, Ulmer et al¹², Wolff et al¹³).

To date, most DNA vaccines in mammalian systems have relied upon viral promoters derived from cytomegalovirus (CMV). These have had good efficiency in both muscle and skin inoculation in a number of mammalian species. A factor known to affect the immune response elicited by DNA immunization is the method of DNA delivery, for example, parenteral routes can yield low rates of gene transfer and produce considerable variability of gene expression¹⁴. High-velocity inoculation of plasmids, using a gene-gun, enhanced the immune responses of mice^{15, 16}, presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

In other embodiments the DNA may be delivered by a virosome or liposome to skin or mucosa. Viral vectors, such as replication-defective adenoviridae, may also be used, and may be delivered by direct, non-invasive (needle-less) vaccination onto bare skins¹⁷.

One of the advantages of a DNA vaccine is that the vector itself can act as an immunologic adjuvant. Firstly, intracellular expression of the antigen allows presentation by the major histocompatibility class I pathway and induction of a CD8+ve cytotoxic T lymphocyte response. Secondly, skewing towards a Th1-response may be enhanced by the use of bacterial DNA as it may contain a high content of unmethylated CpG motifs (e.g. GTCGTT)¹⁸.

In a seventh aspect the present invention consists in a method of inducing an anti-p53 idiotypic response in a subject, the method comprising administering to the subject the composition according to any one of the first, second, third, fourth, fifth or sixth aspects of the invention.

In an eighth aspect the present invention provides a method of inducing immunity against a disease caused by expression of mutant p53, the method comprising administering to the subject the composition according to any one of the first, second, third, fourth, fifth or sixth aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in the specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the nature of the present invention may be more clearly understood preferred forms thereof will be described with reference to the following non-limiting Examples.

Materials and Methods

Preparation of Pentrix

Peptide Manufacture

The production of non-GMP grade peptides (Table 1) was performed by Multiple Peptide Systems (MPS), San Diego, USA. MPS is a specialist unit that routinely produces peptides for numerous clinical trials.

TABLE 1
Peptide sequences of Pentrix ™. These sequences correspond to the
antigen binding regions of two human anti-p53 antibodies with amino (N)
and central domain (C) specificity. The CDR sequences are underlined.
Each peptide contains additional amino acids from the flanking framework
regions from each side of the CDR. CDR = complementarity
determining region.

| | Antibody | Derivation | |
|---|---|---|---|
| | specificity | of CDR | Peptide Sequence |
| 1 | N-terminus | Heavy CDR2 | H-LEWMGIINPSGGSANYAPKFKGRLTMS-OH |
| 2 | N-terminus | Heavy CDR3 | H-AVYYCLLOALKHWGQGT-OH |
| 3 | N-terminus | Light CDR2 | H-KLLIHWASTRESGVPDR-OH |
| 4 | N-terminus | Light CDR3 | H-AGLFCOOYYTTPLTFGGGT-OH |
| 5 | Central | Heavy CDR2 | H-LEWVGFIRSKAYGAATAYAASMKGRVTI-OH |
| | domain | | |
| 6 | Central | Heavy CDR3 | H-YFCSRVKAGGPDYWGQGT-OH |
| | domain | | |
| 7 | Central | Light CDR2 | H-LLIYLGSTRASGVPDR-OH |
| | domain | | |
| 8 | Central | Light CDR3 | H-GVYYCMOGLOTPYTFGEGT-OH |
| | domain | | |

The peptides were sterile with no detectable endotoxin (<0.1 U/mg) and pure as assessed by RP-HPLC and amino add sequence analysis. The vaccine was prepared by admixing the peptides (500 μg of each) with GM-CSF (100 μg; Schering-Plough, Baulkdham Hills, Australia) in 31.25% DMSO/31.25% saline/37.5% dH₂0 (800 μl).

Subjects and Clinical Protocol

Ten subjects with metastatic malignancy (1 breast carcinoma, 5 colorectal carcinoma, 1 non-small cell lung carcinoma, 1 haemangiopericytoma, 1 renal cell carcinoma, 1 prostate carcinoma), 1 subject with local recurrence (squamous cell carcinoma of head and neck) and 3 subjects with definitively treated advanced malignancy who were disease free at study entry (1 esophageal, 1 renal and 1 non-small cell lung cancer) were enrolled onto the trial (median age=52.5 yrs, range 39-70 yrs, 7 males and 7 females). Detailed informed consent was obtained from all of the patients in accordance with the St Vincent's Hospital Human Research Ethics Committee.

Eligible subjects were required to have Eastern Co-operative Oncology Group (ECOG) performance status 0 (11 subjects) or 1 (3 subjects), a life expectancy of at least 6 months, overexpression of p53 in either the primary or metastatic tumour (as indicated by moderate to strong staining of at least 20% of tumour cells with DO7 anti-p53 antibody (Dako, Botany, NSW)) and a positive response to recall antigen as determined by the CMI multitest (Pasteur Merieux, Lyon, France). Subjects were excluded if they had undergone chemotherapy, radiotherapy or surgery or had received immunosuppressive therapy in the preceding 6 weeks.

The first group of subjects received one intradermal vaccination delivered as 4 separate injections of 200 μl. CMI testing of the second group of subjects was preceded by a tetanus booster. The second group then received four vaccinations at monthly intervals, delivered as for the first group. Blood for immunologic assays was drawn before each immunization and 1 and 2 months post-vaccination 4. Clinical observations, including temperature, blood pressure, heart rate and respiratory rate, were recorded at the time of injection and at 24 and 48 hours. Adverse events were graded according to the NCI-Common Toxicity Criteria Version 2¹⁹. Subjects who completed all four vaccinations were assessed for vaccine-specific immune responses. All patients were assessed for safety and toxicity. Tumor response was not assessed as part of this study.

Delayed type hypersensitivity (DTH) testing was carried out 1 month after the final vaccination. The individual vaccine peptides (100 μg), a negative control peptide derived from the light chain CDR2 sequence of a human HIV gp41-specific antibody (H-PKLLIYKASSLESGVPSR-OH) and vehicle only were injected intradermally to the interscapular area. Induration of ≧10 mm² at 48 hours after injection was considered positive.

Immunization of Sheep and Rabbits with the Vaccine

Polyclonal sera specific for each of the peptides were generated by immunizing two sheep and two rabbits. Sheep were primed with 200 μg of the peptide mixture in a CpG DNA adjuvant (ImmunEasy Adjuvant Qiagen), boosted 6 weeks later and bled on the day of priming and 10 days after the boost. Polyclonal sera reactive with peptides 3 and 7 could not be generated in sheep and therefore rabbits were primed with a mixture of peptides 3 and 7 (375 μg each) in a QuilA/DEAE Dextran/Montanide ISA 50V adjuvant mixture (Bioquest, Sydney, Australia) and boosted 6 weeks later.

Humoral Immune Response to the Vaccine and p53

ELISAs were used to detect serum antibodies specific for either the individual vaccine peptides or recombinant p53. Each biotinylated peptide (5 μg/ml; Auspep, Melbourne, Australia) was captured separately onto plates coated with 5 μg/ml of streptavidin (Sigma, St. Louis, Mo.) and the purified recombinant p53 (10 μg/ml) was directly plated as previously described²⁰. Sera were applied in triplicate and tested over a range of dilutions (1:25-1:800). Binding antibodies were detected with an alkaline phosphatase-conjugated goat anti-human IgA+IgG+IgM (H+L) antibody (0.12 μg/ml; Jackson Immunoresearch, West Grove, Pa.). Samples with a mean change in Abs_{410 nm} (the Abs_{410 nm} with peptide—Abs_{410 nm} without peptide) greater than 0.3 were considered positive. Positive responses were isotyped by detecting binding antibodies with a panel of murine anti-human IgA-, IgG1-, IgG2-, IgG3-, IgG4- and IgM specific antibodies (0.25 μg/ml; Zymed, San Francisco, Calif.) followed by a goat anti-mouse IgG alkaline phosphatase conjugate (0.12 μg/ml; Jackson Immunoresearch, West Grove, Pa.). Detection of anti-peptide and anti-p53 responses in the rabbits and sheep were performed using ELISAs as described above, but serum binding antibodies were detected using alkaline phosphatase-conjugated donkey anti-sheep or anti-rabbit IgG antibodies (0.12 μg/ml; Jackson Immunoresearch, West Grove, Pa.). Sera from 54 cancer controls (median age=66.3 yrs, age range=40.9-84.0 yrs, 36 males and 18 females) and 30 normal controls (median age=34.5 yrs, age range=22.7-61.6 yrs, 14 males and 16 females) were also tested.

Cell-Mediated Response to the Vaccine and p53

Proliferation in response to stimulation by vaccine (individual peptides or vaccine pool) or p53 was measured by (³OH)-thymidine and carboxyfluorescein succinimidyl ester (CFSE) proliferation assays, and the secretion of IFN-γ was measured by IFN-γ ELISPOT assay. Peripheral blood mononuclear cells (PBMCs) were plated at a concentration of 1×10⁵ cells/well for thymidine proliferation and ELISpot assays and at 1×10⁶ cells/well (24well plates) for the CFSE proliferation assay. ELISpot plates were coated with mouse anti-human IFN-γ antibody (Diaclone, France) and cells for CFSE assay were stained with 5 μM CFSE-FITC (Molecular Probes, Eugene, Oreg.). All PBMCs were in RPMI supplemented with 2 mM L-glutamine, 10 mM HEPES, 100 U/mL penicillin/streptomycin and 10% human AB serum (Australian Red Cross Blood Service).

Cells were stimulated with the pooled vaccine (50 μg/ml), individual vaccine peptides (10 μg/ml), recombinant p53 (5 or 10 μg/ml), positive controls included phytohaemagglutinin (0.5 μg/ml; Murex, Dartford, UK) and Interleukin-2 (10 U/ml; Roche Diagnostics, Mannheim, Germany) and Staphylococcus enterotoxin B (1 μg/ml; Sigma-Aldrich, St Louis, Mo.) and negative control wells contained medium alone and cell alone. The recall antigens CMV lysate, (1/2000 dilution; BioWhittaker, Walkersville, Md.) and tetanus toxoid (2.5 Lfu/ml; CSL) were also tested. PBMCs were incubated at 37° C. and 5% CO₂ atmosphere for 20 hours (ELISpot) or 6 days (proliferation assays).

IFN-γ secretion was then detected according to the Diadone protocol (Diadone, France), and spots were counted using an automated ELISpot plate counter (Autoimmun Diagnostika, Strassberg, Germany). The frequency of antigen-specific T cells present was calculated by subtracting the mean number of spots obtained in the absence of antigen from the mean number of spots obtained in the presence of antigen. A sample was considered positive if the mean spot count was ≧50 per 10⁶ PBMCs.

In the CFSE proliferation assay, cells were stained with fluorescein-conjugated monoclonal antibodies specific for CD3, CD71 (BD Biosciences, San Jose, Calif.) and CD8 (Beclman Coulter, Miami, Fla.) and analysed using an EPICS-XL flow cytometer²¹. Staining was considered positive when the percentage of cells that were both CD71 positive and had moved out of the undivided population was at least twice that observed in the absence of stimulation.

In the thymidine proliferation assay, cells were pulsed with 0.5 μCi/well [methyl-³H]-thymidine (Amersham, Buckingaamshire, UK) and harvested 18 hours later. [³H]-thymidine incorporation was measured by liquid scintillation counting. A sample was considered to be positive when the stimulation index²² for each well in the quadruplicate sample exceeded the mean of unstimulated cells by at least three standard deviations.

Results

Clinical Outcomes

A total of 102 individuals were screened for entry into this study, forty-two were excluded on clinical grounds and a further 26 individuals were excluded because their tumor did not over-express p53. Twenty-one subjects were subjected to CMI testing and seven of these were excluded due to a lack of demonstrable CMI response to common recall antigens including tetanus toxoid. Four subjects participated in the single-dose phase of the study (subjects 002, 004, 006 and 007) and ten in the multi-dose phase (009, 010, 011, 012, 014, 015, 016, 017, 018 and 019). Four of the multi-dose subjects (009, 010, 011, 019) were withdrawn before they completed all four vaccinations due to disease progression requiring alternative treatment

Over the 49 separate occasions of vaccination, the vaccine was well-tolerated and the majority of recorded adverse events were attributable to the underlying disease. As expected, local reactions were the most common adverse event. Intradermal infiltration of 200 μl resulted in small blebs in all of the subjects, and a number of the early subjects developed blistering and ulceration after 24 to 48 hours. This local reaction was attributed to rapid precipitation of the GM-CSF and peptide mixture and was ameliorated by changing the diluent from saline to water and agitating the syringe between injections. Subjects who received multiple vaccinations developed an increasing area of post-vaccination induration after each occasion, suggestive of a local DTH response.

Toxicities that were probably or possibly attributed to the vaccine were limited to grades 1 or 2 in severity and included arthralgia (10 occasions), nausea (4 occasions) and febrile reactions (16 occasions). These effects have all been described with GM-CSF alone²³. Grade 3 or 4 toxicities, including jaundice (1 occasion), motor neuropathy (2 occasions), vomiting (1 occasion), tumour pain (3 occasions), neuropathic pain (1 occasion) and non-neutropenic sepsis (1 occasion), and two serious adverse events, cord compression and death from pneumonia, were attributable to disease progression.

Humoral Response to the Vaccine

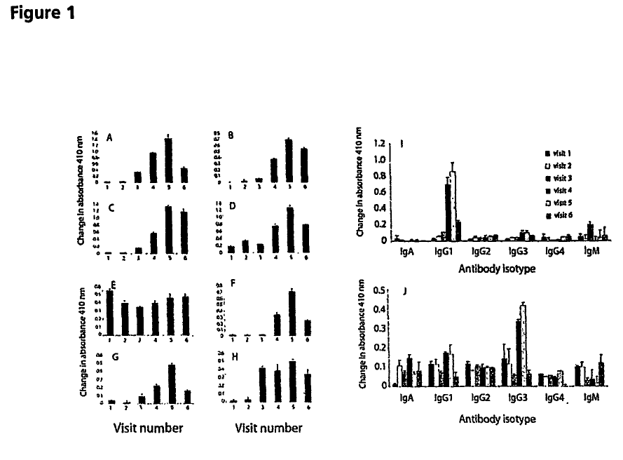

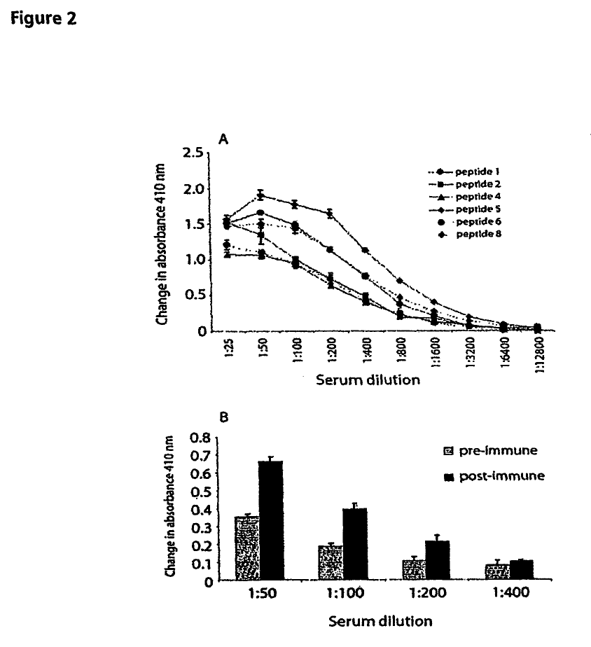

Sera from four of the six assessable trial subjects (012, 014, 015 and 016) were found to be reactive with peptide 2, two (012 and 015) were reactive with peptide 8 and two (015 and 016) were reactive with peptide 5 (FIG. 1A-H). However, one of these responses was observed at the baseline timepoint (patient 015, visit 1) and was not augmented by vaccination (FIG. 1E). No patient demonstrated a response to peptides 1, 3, 4, 6 or 7, although positive responses to each of these peptides were observed using the polyclonal sheep sera (FIG. 2A) or rabbit sera (data not shown). The vaccine-specific humoral responses seen in humans were only detected after at least two vaccinations had been administered. Titres of peptide-specific serum antibodies rose to maximal levels one month after vaccination 4, then fell again in the next month. The humoral responses to the vaccine peptides were further characterized by the use of isotype-specific antibodies. The vaccine-specific serum antibodies in every subject were predominantly of the IgG class, indicative of an antigen-stimulated secondary immune response. Serum titres of the IgG antibodies rose over the period of vaccination in a similar fashion to the combined IgA, IgG and IgM responses. The peptide 2-specific serum antibodies were of the IgG1 subclass in subject 012 (FIG. 1I), IgG1 and IgG3 in subjects 014 and 015 and IgG4 in subject 016 (data not shown). The peptide 5-specific serum antibodies were of the IgG3 subclass in subject 015 (endogenous response) and in subject 016 (FIG. 1J). The peptide 8-specific serum antibodies were only detected at a relatively high serum dilution of 1:25 in subjects 012 and 015 (FIGS. 1G and H) and were undetectable using the isotype-specific antibodies (data not shown).

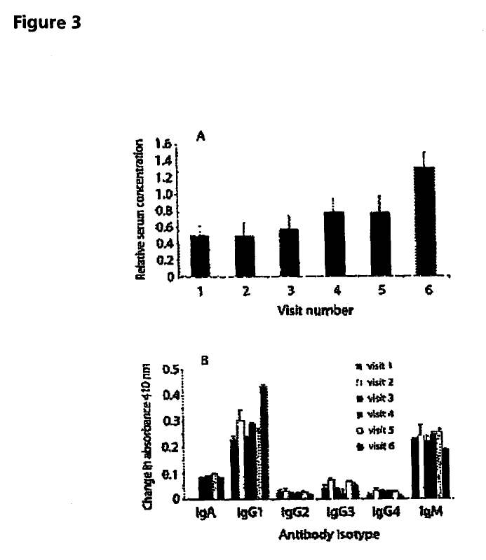

One of the trial subjects (012) also had rising titres of serum anti-p53 antibodies (FIG. 3A). As these antibodies were present at the baseline timepoint, it is not clear whether they were due to the vaccine or were the result of an increased antigen load following disease progression Isotype-specific antibodies showed that the level of p53-specific IgG1 serum antibodies rose with increasing vaccinations, but the level of IgM antibodies remained at a constant level over the course of the vaccination schedule (FIG. 3B).

A small proportion of the sera from the 30 normal and 54 cancer controls demonstrated weak reactivity to peptides in the vaccine although the absorbance in each case was less than that seen in the trial patients (Table 2). Serum reactivity to peptide 1 (3 subjects), peptide 3 (1 subject) and peptide 5 (2 subjects) were detected in the normal control group while responses to peptide 2 (1 subject), peptide 5 (3 subjects) and peptide 7 (1 subject) were seen in the non-trial cancer control population (Table 2). Six of the 54 cancer controls and one of the thirty normal controls had serum antibodies to p53 (Table 2). The p53 mutation status of the cancer controls was unknown.

TABLE 2
Number of individuals with a humoral response to the vaccine peptides
and p53. Sera from the cancer controls and trial subjects were tested
at 1:25 dilution. Trial subjects were examined at a range of concentrations
as shown in the text and FIG. 1. Abbreviations: e endogenous responses
to peptide 5 (subject 015), en endogenous response to p53 (subject 012).
* In the trial subject group, a detectable response at any time point
was considered positive.

| Immunogen | Normal controls | Cancer controls | Trial subjects* |
|---|---|---|---|
| n = 30 | n = 54 | n = 6 | |
| Peptide 1 | 3(10%) | 0 | 0 |
| Peptide 2 | 0 | 1(1.9%) | 4(66.7%) |
| Peptide 3 | 1(3.3%) | 0 | 0 |
| Peptide 4 | 0 | 0 | 0 |
| Peptide 5 | 2(6.7%) | 3(5.6%) | 2e(33.3%) |
| Peptide 6 | 0 | 0 | 0 |
| Peptide 7 | 0 | 1(1.9%) | 0 |
| Peptide 8 | 0 | 0 | 2(33.3) |
| P53 | 1(3.3%) | 6(11.1%) | 1^{en}(16.7%) |

Polyclonal sera with reactivity to peptides 2, 4, 5, 6 and 8 were obtained from both sheep while one animal did not respond to peptide 1 and neither animal responses to peptides 3 and 7 (FIG. 2A). While serum antibody titres were equivalent for peptides 5 and 8, the response to peptide 2, 4, and 6 were on average 50% higher in the first immunized sheep. Given the method of immunization, it was not surprising that the serum anti-peptide titres in sheep were significantly higher than that found in the trial patients. For instance the sheep sera response to peptide 5 was still detectable at serum dilutions of 1:3200 while the highest serum titre in a trial subject was 1:200 to peptide 2 (FIGS. 2A and 1A). Interestingly the sheep with the highest titres of anti-peptide antibodies also had detectable and rising levels of vaccine-induced p53specific antibodies. The p53-specific antibodies were measurable at serum dilutions of 1:50 and 1:100, but then fell to background levels at higher dilutions (FIG. 2B). Polyclonal sera against peptides 3 and 7 were successfully isolated from the immunised rabbits, but neither of the rabbits generated anti-p53 antibodies.

Cell-Mediated Response to the Vaccine

All six of the trial patients who underwent in vivo DTH testing were found to have responses to the individual vaccine peptides (Table 3). The patients each responded to two, three or four of the peptides and responses were observed in at least one individual for all of the peptides, except for peptides 3 and 6. Peptides 5 and 2 were the most immunogenic for T cells with all six of the patients having a peptide 5-specific DTH response and five responding to peptide 2. There were three responses to peptide 8, two to peptides 1 and 7, and one response to peptide 4.

TABLE 3
Extent of dermal induration seen in DTH testing of the six evaluable
patients who completed all four vaccinations. Induration area (m²)
was measured at 48 hours following injection with the individual vaccine
peptides (10 g), HIV-L2 peptide (10 g) and vehicle alone 31.25% dimethyl
sulfoxide (v/v), 0.05 M NaCl. Neg indicates that induration (if any) was
less than 10 mm².
Patient number

| Peptide | 012 | 014 | 015 | 016 | 017 | 018 |
|---|---|---|---|---|---|---|
| Peptide 1 | Neg | Neg | Neg | 104 | 24 | Neg |
| Peptide 2 | 189 | 20 | 86 | Neg | 71 | 38 |
| Peptide 4 | Neg | Neg | 13 | Neg | Neg | Neg |
| Peptide 5 | 165 | 16 | 24 | 269 | 373 | 94 |
| Peptide 7 | 16 | Neg | Neg | 39 | Neg | Neg |
| Peptide 8 | 113 | Neg | 38 | Neg | 44 | Neg |
| HIV-L2 | Neg | Neg | Neg | Neg | Neg | Neg |
| Vehicle | Neg | Neg | Neg | Neg | Neg | Neg |

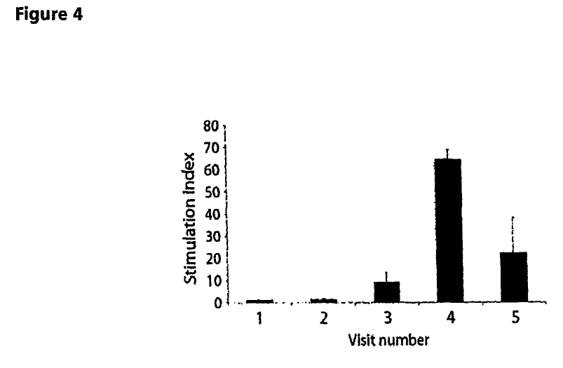

Two of the six assessable trial patients (016 and 017) were found to have T cells specific for the vaccine, as measured by the proliferation assays. Patient 016 showed responses in the thymidine proliferation assay to peptide 1 (visit 5; stimulation index=10.4; data not shown) and peptide 5 (visits 3, 4 and 5; stimulation index=8.88, 64.6 and 22.2, respectively; FIG. 4). This patient also showed a, peptide 5-specific proliferative response first appeared at visit 3 (1 month after-vaccination 2), and increased to maximal levels after three vaccinations had been administered.

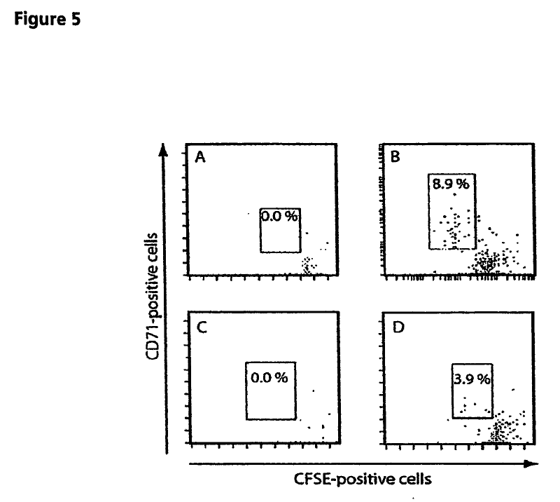

The proliferative response to the vaccine pool was confirmed in subject 016 by CFSE proliferation assay (visit 4; 8.9% of circulating CD3 and CD4 positive cells; FIG. 5B), as was the response to peptide 5 (visit 5; 5.5% of circulating CD3 and CD4 positive cells; data not shown). The other peptides were not tested individually due to limiting cell numbers. Subject 017 also had a specific proliferative response to the vaccine peptide pool measured by CFSE proliferation assay (visit 5; 3.9% of circulating CD3 and CD4 positive cells; FIG. 5D). Both of these vaccine-specific proliferation assays can be attributed to the vaccination schedule as they were not present at baseline timepoints (FIG. 4, FIGS. 5A and 5C). Also, the PBMCs from patients 016 and 017 did not proliferate in the absence of stimulation with either the vaccine pool or peptide 5 (data not shown). Due to limiting cell numbers, only two other patients were tested by CFSE proliferation assay (012 and 015) and neither of them had a measurable vaccine-specific proliferative response.

In contrast to the proliferation assays, none of the trial patients were found to have vaccine-specific IFN-γ secreting T cells measurable by ELISpot assay (data not shown). Five of the patients were tested for responses to CMV lysate and four of the five demonstrated a reproducible specific response to CMV lysate confirming that the cells had been incubated at 37° C. for a sufficient time for a whole protein antigen to be processed and presented in the context of the MHC class I or II pathways. There was no measurable cell-mediated response to p53 in any of the trial subjects as assessed by thymidine proliferation assay, CFSE proliferation assay (only subjects 012, 015, 016 and 017 tested) or IFN-γ ELISpot assay.

None of the cancer controls had a measurable cell-mediated response to the vaccine peptides assayed by thymidine proliferation assay (9 subjects), CFSE proliferation assay (4 subjects) or IFN-γ ELISpot (9 subjects). Similarly, none of the normal controls had a measurable response to p53 assayed by thymidine proliferation assay (3 subjects), or CFSE proliferation assay (2 subjects).

Discussion

In this study we have demonstrated that the CDR regions of human anti-p53 antibodies are capable of initiating humoral and cellular immunity in animals and in individuals with advanced malignancy. Furthermore, anti-p53 antibodies were observed in a sheep and in one individual, suggesting that an idiotype immune cascade may have been triggered by the vaccinations.

A clinically effective immune response to the self-protein p53 requires the production of specific cytotoxic T cells. We have sought to elicit such a response by immunizing with Ab1 (CDR regions from anti-p53 antibodies), and thereby inducing the production of anti-idiotype antibodies (anti-peptide antibodies or Ab2) that are an immunological mimic of p53. In animal models, these anti-idiotype antibodies (Ab2) have been shown to represent a surrogate p⁵³ antigen which can lead to the in vivo production of anti-p53 cytotoxic T cells^{24, 25}. Our strategy differs from previous human idiotype trials in that we have vaccinated subjects with Ab1 rather than anti-idiotype antibodies (Ab2) isolated from immunized animals^{22, 26-29}.

To be effective, the peptide vaccine must first be presented to CD4 positive T cells in the context of the MHC class II pathway. With T cell help, peptide specific B cells must then undergo isotype switching to produce a panel of anti-idiotype (Ab2) antibodies³⁰. Clearly his process has occurred in four of the six evaluable subjects in this study, and by inference the T helper cell response is likely to be of the Th2 subtype. Evidence supporting this contention is the finding of predominantly IgG1 and IgG3 anti-peptide antibodies, and the identification of vaccine-specific T cells which failed to secrete IFN-γ. Interestingly, only three of the eight peptides (2, 5 and 8) were responsible for the observed humoral and cell mediated immune response. There are a number of possible explanations for the lack of immunogenicity of the remaining peptides, including the format and timing of vaccinations, and their HLA compatibility with the subjects in this study³¹⁻³³. One of the most likely reasons is that since the peptides are derived from entirely human immunoglobulin sequences, the particular T and B cells responsible for recognition of these self sequences may have been deleted during clonal selection³⁴. Certainly the studies of Ruiz and colleagues have demonstrated that only a subset of the mouse anti-p53 CDRs are immunogenic in mouse models^{24, 25}.

Given that vaccinated subjects were able to produce antigen-specific Ab2, we hypothesized that a proportion of these Ab2 molecules could trigger an anti-p53 (Ab3) response. Indeed this was the case in one of two vaccinated sheep, and in one of the patients, as demonstrated by rising titres of p53-specific antibodies. With regard to the sheep, the p53-specific response was clearly induced by the vaccine, since it was not detectable in the pre-immune serum, and correlated well with the high titres of vaccine-specific antibodies. On the other hand, the significance of the anti-p53 response in the trial subject remains uncertain. This individual had an endogenous p53-specific immune response at baseline, and while the titre of anti-p53 antibodies increased over the course of the vaccinations, the tumor burden was also increasing over this period.

On the basis of these findings, it seems reasonable to conclude that the current vaccination strategy was not optimal in inducing detectable anti-p53 immune responses, at least in humans. There are a number of explanations for this finding which warrant further consideration. Firstly, it is important to consider the possibility that the in vitro assays used in this study failed to detect humoral and CD8+ T cell responses to p53. While this seems unlikely, it is true that reactivity to conformational epitopes on recombinant p53 would not be detected in the ELISA. Furthermore, in the absence of cross priming, the T cell assays would only identify CD4+ responses, since the p53 was added exogenously and presented in association with MHC class II by antigen presenting cells. Detection of a CD8 positive response to p53 would require the in vitro use of short overlapping peptides (9 to 13 amino acids in length) which span the length of p53³⁵.

These technical issues aside, it is important to identify those characteristics of Ab2 which will produce anti-p53 antibodies (Ab3). In this regard, we suggest that the absolute amount of Ab2 available for antigen presentation may impact on its potential role as an immunogen. The highest titers of Ab2 were observed in the sheep with an anti-p53 antibody response. The second sheep, which had much lower titers of vaccine-specific antibodies, correspondingly showed no detectable p53-specific humoral immunity. While the titers of Ab2 in the trial patients were many fold lower than those seen in either sheep, it is interesting to note that the patient with the highest Ab2 response was also the individual in whom there was demonstrable anti-p53 reactivity.

Another factor which may have influenced the development of Ab3 was the time taken to develop anti-peptide or Ab2 responses a factor that will in turn be dependent upon the duration of vaccination. Evaluable patients did not develop peptide immunity until after the third or fourth vaccination, and the titer of antibody fell once vaccinations were discontinued. It is therefore possible that anti-p53 antibodies may only be generated following repeated vaccinations over a sustained period of time. Comparison with other vaccine trials suggests that peptides are best administered at weekly intervals for 4 weeks, followed by regular boosts over a period of many months³⁶⁻³⁸. Indeed since p53 is self protein, immune responses are unlikely to have good memory and therefore vaccinations may need to be continued indefinitely.

A final possibility which may explain the failure to induce anti-p53 antibodies is that the epitopes on Ab2 may mimic regions of p53 which are not recognised by the immune system. Since the idiotype network is designed to generate topochemical copies of antigenic epitopes³⁹, and the vaccine was derived from individuals with specific human anti-p53 antibodies, this explanation seems less likely.

Irrespective of the mechanism involved, in order to avoid difficulties that may be encountered in production of sufficient the levels of vaccine-specific antibodies an alternative is to isolate and immunize with vaccine-specific antibodies (Ab2), thus eliminating one step in the idiotype cascade. A further advantage of this approach is that it will allow the design of vaccines which immediately elicit the production of anti-p53 antibodies, and more importantly CD8+ T cell cells^{40, 41}. Considering the potential time taken to complete all steps of the idiotype cascade, further immunization with Ab1 may not be prudent, especially in the adjuvant setting where the induction of anti-tumor immunity immediately following surgery is important.

In conclusion, this study demonstrates that vaccinating with human antibody CDR regions represents a novel method for inducing human Ab2, and in turn suggests that isolation of these antibodies could yield a useful immunogen, particularly in the adjuvant setting.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Donnelly et al, Journal of Immunological Methods 176 (1994) 145-152.
2. Conry et al., Cancer Res 54:1164-1168, 1994.
3. Cardoso et al., Immuniz Virol 225:293-299, 1996.
4. Cox et al., J Virol 67:5664-5667, 1993.
5. Davis et al., Hum Mol Genet 2:1847-1851, 1993.
6. Sedegah et al., Proc Natl Acad Sci USA 91:9866-9870, 1994.
7. Montgomery et al., DNA Cell Biol 12:777-783, 1993.
8. Ulmer et al., Science 259:1745-1749, 1993.
9. Wang et al., Proc Natl Acad Sci USA 90:4156-4160, 1993.
10. Xiang et al., Virology 199:132-140, 1994.
11. Yang et al., Vaccine 15:888-891, 1997.
12. Ulmer et al Science 259:1745, 1993.
13. Wolff et al Biotechniques 11:474, 1991.
14. Montgomery et al, DNA Cell Biol 12:777-783, 1993.
15. Fynan et al., Proc Natl Acad Sci USA 90:11478-11482, 1993.
16. Eisenbraun et al., DNA Cell Biol 12:791-797, 1993.
17. Shi Z, Vaccine 17:2136-2141, 1999.
18. Ada G, NEJM, 345: 1042-1053.
19. http://ctep.cancer.gov/forms/CTCv20 4-30-992.pdf.
20. Coomber, D. W. J., Hawkins, N. J., Clark, M. A., and Ward, R. L. Generation of anti-p53 Fab fragments from individuals with colorectal cancer using phage display. Journal of immunology. 163: 2276-2283, 1999.
21. Lyons, A. B. Analysing cell division in vivo and in vitro using flow cytometric measurement of CFSE dye dilution. Journal of immunological Methods. 243: 147-154, 2000.
22. Foon, K. A., John, W. J., and Chakraborty, M. J. Clinical and immune responses in resected colon cancer patients treated with anti-idiotype monoclonal antibody vaccine that mimics the carcionembryonic antigen. Journal of Clinical Oncology. 17:2889-2895, 1999.
23. McNeel, D. G., Schiffman, K., and Disis, M. L. Immunisation with recombinant human granulocytemacrophage colony-stimulating factor as a vaccine adjuvant elicits both a cellular and humoral response to recombinant human granulocyte-macrophage colony-stimulating factor. Blood. 93:2653-2659, 1999.
24. Erez-Alon, N., Herkel, J., Wolkowicz, R., Ruiz, P. J., Waisman, A., Rotter, V., and Cohen, I. R. Immunity to p53 induced by an idiotypic network of anti-p53 antibodies:generation of sequence-specific anti-DNA antibodies and protection from tumour metastasis. Cancer Research. 58:5447-5452, 1998.
25. Ruiz, P. J., Wolkowicz, R. Waisman, A., Hirscbberg, D. L., Carmi, P., Erez, N., Garren, H., Herkel, J., Karpuj, M., Steinman, L., Rotter, V., and Cohen, I. R. Idiotypic immunisation induces immunity to mutated p53 and tumour rejection. Nature Medicine. 4:710-712, 1998.
26. Foon, K. A., John, W. J., Chakraborty, M., Sherratt, A., Garrison, J., Flett, M., and Bhattacharya-Chattezjee, M. Clinical and immune responses in advanced colorectal cancer patients treated with anti-idiotypic monoclonal antibody vaccine that mimics the carcinoembryonic antigen. Clinical Cancer Research. 3: 1267-1276, 1997.
27. Foon, K. A., Oseroff, A. R., Vaickus, L., Greenberg, S. J., Russell, D., Bernstein, Z., Pincus, S., Kohler, H., Seon, B. K., Tahaoglu, E., Beers, T., Chakraborty, M., and Bhatacharya-Chatterjee, M. Immune responses in patients with T-cell lymphoma treated with an anti-idiotype antibody mimicking a highly restricted T-cell antigen. Clinical Cancer Research. 1:1285-1294, 1995.
28. Foon, K. A., Lutzky, J., Baral, R. N., Yannelli, J. R., Hutchins, L., Teitelbaum, A., Kashala, O. L., Das, R., Garrison, J., Reisfeld, R. A., and Bhattacharya-Chatterjee, M. Clinical and immune responses in advanced melanoma patients immunized with am anti-idiotype antibody mimicking disialoganglioside GD2. Journal of Clinical Oncology. 18.376-384, 2000.
29. Somasundaram, R., Zaloudik, J., Jacob, L., Benden, A., Sperlagh, M., Hart E., Marks, G., Kane, M., Mastrangelo, M., and Herlyn, D. Induction of antigen-specific T and B cell immunity in colon carcinoma patients by anti-idiotypic antibody. J Immunol. 155:3253-3261, 1995.
30. 42. Foon, K. A., Yannelli, J., and Bhattacharya-Chattezjee, M. Colorectal cancer as a model for immunotherapy. Clin Cancer Res. 5:225-236, 1999.
31. Disis, M. L. and Schiffman, K. Issues on Clinical Applications of Cancer Vaccines. J Immunother. 24:104-105, 2001.
32. Monzavi-Karbassi, B. and Kieber-Emmons, T. Current concepts in cancer vaccine strategies. Biotechniques. 30:170-172, 174, 176 passim, 2001.
33. Machiels, J. P., van Baren, N., and Marchand, M. Peptide-based cancer vaccines. Sernin Oncol. 29:494-502, 2002.
34. Keilholz, U., Weber, J., Finke, J. H., Gabrilovich, D. I., Kast, W. M., Disis, M. L., Kirkwood, J. M., Scheibenbogen, C., Schlom, J., Maino, V. C., Lyerly, H. K., Lee, P. P., Storkus, W., Marincola, F., Worobec, A., and Atkins, M. B. Immunologic monitoring of cancer vaccine therapy: results of a workshop sponsored by the Society for Biological Therapy. J Immnunother. 25:97-138, 2002.
35. Theobald, M., Biggs, J., Dittmer, D., Levine, A. J., and Sherman, L. A. Targeting p53 as a general tumor antigen. Proc Natl Acad Sci USA. 92:11993-11997, 1995.
36. Jager, E., Ringhoffer, M., Dienes, H. P., Arand, M., Karbach, J., Jager, D., Ilsemann, C., Hagedom, M., Oesch, F., and Knuth, A. Granulocyte-macrophage-colony-stimulating factor enhances immune responses to melanoma-associated peptides in vivo. Int J Cancer. 67:54-62, 1996.
37. Disis, M. L., Grabstein, K. H., Sleath, P. R., and Cheever, M. A. Generation of immunity to the HER-2/neu oncogenic protein in patients with breast and ovarian cancer using a peptide-based vaccine. Clin Cancer Res. 5:1289-1297, 1999.
38. Gjersten, M. K., Buanes, T., Rosseland, A. R., Bakka, A., Gladhaug, I., Soreide, O., Eriksen, J. A., Moller, M., Baksaas, I., Lothe, R. A., Saeterdal, I., and Gaudemack, G. Intradermal Ras peptide vaccination with granulocyte-macrophage colony-stimulating factor as adjuvant: clinical and immunological responses in patients with pancreatic adenocarcinoma. International Journal of Cancer. 92:441-450, 2001.
39. Bona, C. A. Idiotype vaccines: forgotten but not gone. Nat Med. 4:668-669, 1998.
40. Moingeon, P., Haensler, J., and Lindberg, A. Towards the rational design of Th1 adjuvants. Vaccine. 19:4363-4372, 2001.
41. Moingeon, P. Strategies for designing vaccines eliciting Th1 responses in humans. Journal of Biotechnology. 98:189-198, 2002.

1. An idiotypic vaccine composition, the vaccine composition comprising a pharmaceutically acceptable carrier and at least one peptide, wherein the at least one peptide is selected from the group consisting of X₁-LLQALKH (SEQ ID NO: 1)-Y₁, X₂-FIRSKAYGAATAYAASMKG (SEQ ID NO: 2)-Y₂ and X₃-MQGLQTPYT (SEQ ID NO: 3)-Y₃ in which X₁, X₂, X₃, Y₁, Y₂ and Y₃ are independently either absent or an amino acid sequence of preferably less than 10 amino acids which provides a framework for the specified peptide.

2. The composition according to claim 1 wherein X₁ is absent or is AVYYC (SEQ ID NO: 7), X₂ is absent or is LEWVG (SEQ ID NO: 8), X₃ is absent or is GVYYC (SEQ ID NO: 9), Y₁ is absent or is WGQGT (SEQ ID NO: 10), Y₂ is absent or is RVTI (SEQ ID NO: 11) and Y₃ is absent or is FGEGT (SEQ ID NO: 12).

3. The composition according to claim 1 or 2 wherein at least one peptide is selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

4. The composition according claim 1 wherein the composition comprises at least 2 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

5. The composition according to claim 1 wherein the composition comprises all 3 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

6. The composition according to claim 1 wherein the composition further comprises at least one peptide selected from the group consisting of LEWMGIINPSGGSANYAPKFKGRLTMS (SEQ ID NO: 13), KLLIHWASTRESGVPDR (SEQ ID NO: 14), AGLFCQQYYTTPLTFGGGT (SEQ ID NO: 15), YFCSRVKAGGPDYWGQGT (SEQ ID NO: 16) and LLIYLGSTRASGVPDR (SEQ ID NO: 17).

7. The composition according to claim 1 wherein the peptide is an analogue of a peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO. 6).

8. The composition according to claim 1 wherein the composition further comprises an adjuvant.

9. The composition according to claim 8 wherein the adjuvant is selected from the group consisting of cytokines, immune stimulatory complexes (ISCOMS), CpG oligonucleotides, lipopolysaccharide, muramyl dipeptides, bacterial toxins such as diptheria, pertussis and tetanus toxins, ovalbumin, N-acetylmuramyl-L-alanyl-D-isoglutaminyl-L-alanine-2-(1′-2′-dipalmit-oyl-sn-glycero-3-hydroxyphosphoryloxy)-ethylamine, RIBI, aluminium potassium sulfate (alum), beryllium sulfate, silica, kaolin, carbon, water-in-oil emulsions, oil-in-water emulsions, Corynebacterium parvum, Bordetella pertussis, polyribonucleotides, sodium alginate, lanolin, lysolecithin, vitamin A, saponin, liposomes, levamisole, DEAE-dextran, blocked copolymers or other synthetic adjuvants including Merck Adjuvant 65, or Freund's Incomplete Adjuvant and Complete Adjuvant.

10. The composition according to claim 9 wherein the adjuvant is granulocyte-macrophage colony stimulating factor (GM-CSF).

11. An idiotypic vaccine composition, the vaccine comprising a pharmaceutically acceptable carrier and at least one peptide, the at least one peptide being characterised in that it competes with a peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6) for binding to p53.

12. An idiotypic vaccine composition, the composition comprising a pharmaceutically acceptable carrier and at least one peptide, the at least one peptide being characterised in that an antibody raised against the peptide reacts with at least one peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

13. The composition according to claim 11 or 12 wherein the peptide is derived from the complementarity determining region of a human anti-p53 antibody.

14. The composition according to claim 11, wherein the at least one peptide is selected from the group consisting of X₁-LLQALKH (SEQ ID NO: 1)-Y₁, X₂-FIRSKAYGAATAYAASMKG (SEQ ID NO: 2)-Y₂ and X₃-MQGLQTPYT (SEQ ID NO: 3)-Y₃ in which X₁, X₂, X₃, Y₁, Y₂ and Y₃ are independently either absent or an amino acid sequence of preferably less than 10 amino acids which provides a framework for the specified peptide.

15. The composition according to claim 14 wherein X₁ is absent or is AVYYC (SEQ ID NO: 7), X₂ is absent or is LEWVG (SEQ ID NO: 8), X₃ is absent or is GVYYC (SEQ ID NO: 9), Y₁ is absent or is WGQGT (SEQ ID NO: 10), Y₂ is absent or is RVTI (SEQ ID NO: 11) and Y₃ is absent or is FGEGT (SEQ ID NO: 12).

16. The composition according to claim 11 wherein the at least one peptide is selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

17. The composition according to claim 11 wherein the composition comprises at least 2 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

18. The composition according to claim 11 wherein the composition comprises all 3 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

19. The composition according to claim 11 wherein the composition further comprises at least one peptide selected from the group consisting of LEWMGIINPSGGSANYAPKFKGRLTMS (SEQ ID NO: 13), KLLIHWASTRESGVPDR (SEQ ID NO: 14), AGLFCQQYYTTPLTFGGGT (SEQ ID NO: 15), YFCSRVKAGGPDYWGQGT (SEQ ID NO: 16) and LLIYLGSTRASGVPDR (SEQ ID NO: 17).

20. The composition according to claim 11 wherein the peptide is an analogue of a peptide selected from the group consisting of AVYYCLLQALKHWGQGT (SEQ ID NO: 3), LEWVGFIRSKAYGAATAYAASMKGRVTI (SEQ ID NO: 4), and GVYYCMQGLQTPYTFGEGT (SEQ ID NO: 6).

21. The composition according to claim 11 wherein the composition further comprises an adjuvant.

22. The composition according to claim 21 wherein the adjuvant is selected from the group consisting of cytokines, immune stimulatory complexes (ISCOMS), CpG oligonucleotides, lipopolysaccharide, muramyl dipeptides, bacterial toxins such as diptheria, pertussis and tetanus toxins, ovalbumin, N-acetylmuramyl-L-alanyl-D-isoglutaminyl-L-alanine-2-(1′-2′-dipalmit-oyl-sn-glycero-3-hydroxyphosphoryloxy)-ethylamine, RIBI, aluminium potassium sulfate (alum), beryllium sulfate, silica, kaolin, carbon, water-in-oil emulsions, oil-in-water emulsions, Corynebacterium parvum, Bordetella pertussis, polyribonucleotides, sodium alginate, lanolin, lysolecithin, vitamin A, saponin, liposomes, levamisole, DEAE-dextran, blocked copolymers or other synthetic adjuvants including Merck Adjuvant 65, or Freund's Incomplete Adjuvant and Complete Adjuvant.

23. The composition according to claim 22 wherein the adjuvant is granulocyte-macrophage colony stimulating factor (GM-CSF).

24. An idiotypic vaccine composition, the vaccine comprising a pharmaceutically acceptable carrier and at least one DNA molecule, the DNA molecule comprising a sequence encoding at least one peptide, wherein the at least one peptide is selected from the group consisting of X₁-LLQALKH (SEQ ID NO: 1)-Y₁, X₂-FIRSKAYGAATAYAASMKG (SEQ ID NO: 2)-Y₂ and X₃-MQGLQTPYT (SEQ ID NO: 3)-Y₃ in which X₁, X₂, X₃, Y₁, Y₂ and Y₃ are independently either absent or an amino acid sequence of preferably less than 10 amino acids which provides a framework for the specified peptide.

25. The composition according to claim 24 wherein X₁ is absent or is AVYYC (SEQ ID NO: 7), X₂ is absent or is LEWVG (SEQ ID NO: 8), X₃ is absent or is GVYYC (SEQ ID NO: 9), Y₁ is absent or is WGQGT (SEQ ID NO: 10), Y₂ is absent or is RVTI (SEQ ID NO: 11), Y₃ is absent or is FGEGT (SEQ ID NO: 12).

26. The composition according to claim 24 or 25 wherein the composition comprises a DNA molecule encoding at least one peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

27. The composition according to claim 24 wherein the composition comprises least 2 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

28. The composition according to claim 24 wherein the composition comprises all 3 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

29. The composition according to claim 24 wherein the DNA molecule comprises a further sequence encoding GM-CSF.

30. The composition according to claim 24 wherein the DNA molecule comprises a further sequence encoding LEWMGIINPSGGSANYAPKFKGRLTMS (SEQ ID NO: 13), KLLIHWASTRESGVPDR (SEQ ID NO: 14), AGLFCQQYYTTPLTFGGGT (SEQ ID NO: 15), YFCSRVKAGGPDYWGQGT (SEQ ID NO: 16) and LLIYLGSTRASGVPDR (SEQ ID NO: 17).

31. The composition according to claim 24 for use in DNA vaccination.

32. An idiotypic vaccine composition, the vaccine comprising a pharmaceutically acceptable carrier and at least one DNA molecule, the DNA molecule comprising a sequence encoding at least one peptide, the at least one peptide being characterised in that it competes with a peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6). for binding to p53.

33. An idiotypic vaccine composition, the composition comprising a pharmaceutically acceptable carrier and at least one DNA molecule, the DNA molecule comprising a sequence encoding at least one peptide, the at least one peptide being characterised in that an antibody raised against the peptide reacts with at least one peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

34. The composition according to claim 32 or 33 wherein the peptide encoded by the DNA molecule is derived from the complementarity determining region of a human anti-p53 antibody.

35. The composition according to claim 32 wherein the peptide encoded by the DNA molecule is selected from the group consisting of X₁ is absent or is AVYYC (SEQ ID NO: 7), X₂ is absent or is LEWVG (SEQ ID NO: 8), X₃ is absent or is GVYYC (SEQ ID NO: 9), Y₁ is absent or is WGQGT (SEQ ID NO: 10), Y₂ is absent or is RVTI (SEQ ID NO: 11), Y₃ is absent or is FGEGT (SEQ ID NO: 12), in which X₁, X₂, X₃, Y₁, Y₂ and Y₃ are independently either absent or an amino acid sequence of preferably less than 10 amino acids which provides a framework for the specified peptide.

36. The composition according to claim 35 wherein X₁ is absent or is AVYYC (SEQ ID NO: 7), X₂ is absent or is LEWVG (SEQ ID NO: 8), X₃ is absent or is GVYYC (SEQ ID NO: 9), Y₁ is absent or is WGQGT (SEQ ID NO: 10), Y₂ is absent or is RVTI (SEQ ID NO: 11), Y₃ is absent or is FGEGT (SEQ ID NO: 12).

37. The composition according to claim 32 wherein the composition comprises a DNA molecule encoding at least one peptide selected from the group consisting of AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

38. The composition according to claim 32 wherein the composition comprises least 2 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

39. The composition according to claim 32 wherein the composition comprises all 3 of the peptides AVYYCLLQALKHWGQGT (SEQ ID NO: 4), LEWVGFIRSKAYGAATAYAASMKGRVTI (SEQ ID NO: 5), and GVYYCMQGLQTPYTFGEGT (SEQ ID NO: 6).

40. The composition according to 32 wherein the DNA molecule comprises a further sequence encoding GM-CSF.

41. The composition according to claim 32 wherein the DNA molecule comprises a further sequence encoding LEWMGIINPSGGSANYAPKFKGRLTMS (SEQ ID NO: 13), KLLIHWASTRESGVPDR (SEQ ID NO: 14), AGLFCQQYYTTPLTFGGGT (SEQ ID NO: 15), YFCSRVKAGGPDYWGQGT (SEQ ID NO: 16) and LLIYLGSTRASGVPDR (SEQ ID NO: 17).

42. The composition according to claim 32 for use in DNA vaccination.

43. A method of inducing an anti-p53 idiotypic response in a subject, the method comprising administering to the subject the composition according to claim 1.

44. A method of inducing immunity against a disease caused by expression of mutant p53, the method comprising administering to the subject the composition according to claim 1.


SEQUENCE LISTING (17 sequences)

SEQ ID NO: 1 - protein, 7 aa, Artificial
  incorporating a CDR of an anti-p53 antibody
LLQALKH

SEQ ID NO: 2 - protein, 19 aa, Artificial
  incorporating a CDR of an anti-p53 antibody
FIRSKAYGAATAYAASMKG

SEQ ID NO: 3 - protein, 9 aa, Artificial
  incorporating a CDR of an anti-p53 antibody
MQGLQTPYT

SEQ ID NO: 4 - protein, 17 aa, Artificial
  incorporating a CDR of an anti-p53 antibody
AVYYCLLQALKHWGQGT

SEQ ID NO: 5 - protein, 28 aa, Artificial
  incorporating a CDR of an anti-p53 antibody
LEWVGFIRSKAYGAATAYAASMKGRVTI

SEQ ID NO: 6 - protein, 19 aa, Artificial
  incorporating a CDR of an anti-p53 antibody
GVYYCMQGLQTPYTFGEGT

SEQ ID NO: 7 - protein, 5 aa, Artificial
  incorporating a CDR of an anti-p53 antibody
AVYYC

SEQ ID NO: 8 - protein, 5 aa, Artificial
  incorporating a CDR of an anti-p53 antibody
LEWVG

SEQ ID NO: 9 - protein, 5 aa, Artificial
  incorporating a CDR of an anti-p53 antibody
GVYYC

SEQ ID NO: 10 - protein, 5 aa, Artificial
  incorporating a CDR of an anti-p53 antibody
WGQGT

SEQ ID NO: 11 - protein, 4 aa, Artificial
  incorporating a CDR of an anti-p53 antibody
RVTI

SEQ ID NO: 12 - protein, 5 aa, Artificial
  incorporating a CDR of an anti-p53 antibody
FGEGT

SEQ ID NO: 13 - protein, 27 aa, Artificial
  incorporating a CDR of an anti-p53 antibody
LEWMGIINPSGGSANYAPKFKGRLTMS

SEQ ID NO: 14 - protein, 17 aa, Artificial
  incorporating a CDR of an anti-p53 antibody
KLLIHWASTRESGVPDR

SEQ ID NO: 15 - protein, 19 aa, Artificial
  incorporating a CDR of an anti-p53 antibody
AGLFCQQYYTTPLTFGGGT

SEQ ID NO: 16 - protein, 18 aa, Artificial
  incorporating a CDR of an anti-p53 antibody
YFCSRVKAGGPDYWGQGT

SEQ ID NO: 17 - protein, 16 aa, Artificial
  incorporating a CDR of an anti-p53 antibody
LLIYLGSTRASGVPDR